(19) United States
(12) Patent Application Publication
(10) Pub. No.: US 2005/0106276 A1
(43) Pub. Date: May 19, 2005

(54) Pharmaceutical composition made from Chinese traditional medecine and method of producing thereof

(75) Inventor: Wei Xiao, Jiangsu (CN)

Correspondence Address: BANNER & WITCOFF, 1001 G STREET N W, SUITE 1100, WASHINGTON, DC, 20001, US

(73) Assignee: Jiangsu Kanion Pharmaceutial Co. Ltd., Jiangsu (CN)

(21) Appl. No.: 10/497,032

(22) PCT Filed: Nov. 29, 2001
(86) PCT No.: PCT/CN01/01593

(51) Int. Cl. A61K 35/78

(52) U.S. Cl. 424/773; 424/779; 424/777

(57) ABSTRACT

This invention has published a composition of medicine and its preparation, which can treat diabetes, hyperlipemia, fatigue and hypoxia. The preparation is as following: 300-1000 part-by-weight of prepared rehmannia root, 150-600 part-by-weight of prepa red dogwood fruit, 100-450 part-by-weight of tree peony bark, 150-600 part-by-weight of chinese yam, 100-450 part-by-weight of indian bread, 100-450 part-by-weight of alisma rhizome, right amount of refined edible vegetable oil are the materials; the tree peony bark is distilled; the residue of the tree peony bark after distillation, the prepared rehmannia root, the chinese yam and the indian bread are decocted to get the mixed liquor; the paeonol mother liquor is added into the mixed liquor to get extracturm I by centrifugation and concentration; the prepared dogwood fruit and the alisma rhizome are extracted in the refluxing alcohol; the extract is concentrated to get extracturm II; the extracturm I and II are mixed and concentrated to the thick paste; the thick paste is dehydrated in the vacuum, smashed and sifting; The refined paeonol is smashed and added into the extracturm powder; the mixed powder is added with right amount of supplementary materials and milled by the colloid mill, sifting screening; the fine powder is made into soft capsule. The invented capsule has the features of bringing into play in a shorter time, better curative effect and lower dose for oral administration, easier to administrate and stable product quality.

### FIELD OF THE INVENTION

This invention is about a composition of medicine extracted from herbs and the preparation method of it.

### BACKGROUD OF THE INVENTION

Kidney is the basis congenital constitution in the theory of Chinese medicine, where preserving the primordial Yin and Yang. It is important to keep but not release the element of the kidney, because it is the basis of Zang-fu Yin and Yang. Congenital deficiency, improper regimen, prolonged diseases or genuine energy deficit while aged all can lead the deficiency of the kidney syndrome. The kidney-yin is the basis of body yin liquid, have the functions of nourishing and moistening Zang-fu, nurturing brain marrow and bones, so that if the kidney-yin was impairment, can't nourishing and asthenic fever from interior can lead the deficiency of the kidney syndrome. Prepared rehmannia root is the processed drugs of rehmannia root belong to figwort family. Mainly producing areas is Henan and Zhejiang province, sweet and lukewarm nature, belong to liver and kidney channels, with the function of nourishing yin and supplementing blood and invigorating genuine energy and nurturing brain marrow. Essential component is iridoid glycosides such as catalpol, mannitol, rehmannin etc. Pharmacological testing showed it have the notable effect of tonifying the heart, boosting pressure, inducing diuresis and degrade the blood sugar level. Asiatic Cornelian Cherry Fruit is the dry pulp of the mature Asiatic Cornelian Cherry belongs to Dogwood family. Mainly producing areas is Henan and Shanxi province. Sour, astringent and lukewarm nature, belong to liver and kidney channels, with the function of tonifying the liver and kidney and arresting seminal emission. Essential components are verbenalin, saponin, ursolic and Aleppo gall acid etc. Pharmacological testing showed it have the effect of anti inflammatory, degrade the blood sugar level, antishock and tonifying the heart. Common Yan Rhizome is the dry rhizomes of cinnamonvine belong to Dioscoreaceae. Mainly producing areas is Henan and Hebei province, sour, sweet and lukewarm nature, belong to spleen, stomach and liver channel, with the function of reinforcing the spleen, nourishing the stomach, promoting the production of the body fluid, benefiting the lung, tonifying the kidney and arresting seminal emission. Essential components are saponin, bilineurin, arginine and amylum. Pharmacological testing showed it have the effect of tonifying and strengthening, help digest and immunization. Oriental water plantain rhizome is the dry tuber of Oriental water plantain rhizome belongs to Alismaceae. Mainly producing areas is Fujian, Jiangxi and Sichuan province, sweet and cold nature, belong to kidney and bladder channel, with the function of inducing diuresis and dispersing damp-heat, Essential components are alismol A, B, C and alismol A acetas, alismol B acetas, alismol C acetas, bilineurin and lecithin. Pharmacological testing showed it have the effect of inducing diuresis, reduce blood fat and anti-fatty liver etc. Tree peony Bark is the dry root bark of peony belong to Ranunculaceae, Mainly producing areas is Henan and Anhui province. Bitter, acrid and little cold nature, belong to heart, liver and kidney channels, with the function of removing pathogenic heat from blood and promote blood circulation to dispel blood stasis. Essential components are paeonol, paeoniflorin and aetherolea. Pharmacological testing showed it have the effect of receding the blood pressure, mitigate, hypnosis, relieve pain, anti convulsion, relieve fever, anti inflammatory, anti anaphylaxis and bacteriostasis etc. Indian Bread is the dry sclerote of Indian Bread eumycete belong to polyporaceae Mainly producing areas is Shanxi and Anhui province, sweet and light nature, belong to heart, lung, spleen and kidney channel, with the function of removing dampness and promoting diuresis, invigorating the spleen and calming the mind. Essential components are tumulosic acid, pachyman, tuber acid etc. Pharmacological testing showed it have the effect of inducing diuresis and bacteriostasis etc.

### SUMMARY OF THE INVENTION

The invention is aimed to provide a composition of medicine for treating the damage of kidney-YIN, dizziness and tinnitus, soreness of the waist and the knees, osteopyrexia and fever, night sweat, emissions and diabetes etc. It was extracted from herbs and with the character such as fast assimilates, good curative effect, no side effect and convenient taking; we also provide the preparative method and new usage of it.

The purpose of this invention is implemented thorough follow technique project:

| | |
|---|---|
| Prepared Rehmannia Root | 300-1000 |
| part-by-weight | |
| Prepared Asiatic Cornelian Cherry Fruit | 150-600 |
| part-by-weight | |
| Tree peony Bark | 100-450 |
| part-by-weight | |
| Common Yan Rhizome | 150-600 |
| part-by-weight | |
| Indian Bread | 100-450 |
| part-by-weight | |
| Oriental Waterplantain Rhizome | 100-450 |
| part-by-weight | |
| Refined edible vegetable oil | sufficient quantum |

The Tree peony Barks were moistened 1-3 hours with 6-12 times water, distill extracting it 6-12 hours with vapor, then collected distillate 8 times of the Tree peony Bark, keep mother liquid alone; congealing the distillate 10-14 hours at 0-4° C., leached paeonol, dried it with cold wind, airtight preserve and keep alone; Mixing above residue, Prepared Rehmannia Root, Common Yan Rhizome and Indian bread, adding 6-10 times water, then cooking the mixture 1-4 times, each time 0.5-2 hours, combinging all the cooked water and the mother liquid of paeonol together, then concentrate it to the extractum with d=1.10 at 80° C., centrifugated with centrifugal separator on the speed of 20000 r/m for 20 minutes, Discard the residue after centrifugated then condensing to the extractum I with relative density is 1.13.

The Asiatic Cornelian Cherry Fruit and Oriental Waterplantain Rhizome were Crushed to coarse powder, adding 50-80% alcohol 4-8 times of it, then extracted it 1-3 times, each time is 2-4 hours, combined the filtrate, retrieving the alcohol, condensing to extractum II with relative density is 1.14 at 80° C.; combined extractum I and extractum II, condensed to thick paste with relative density is 1.30-1.35 at 80° C.; then vacuum drying, crushing and sieving. Crushing Paeonol and mingling with the powdered extract, adding proper amount supplementary materials, then grinded it with colloid mill, sieving, pressing to get elastic capsule, each capsule containing 1.0 g, approximately 3.0 g crude drug; or each capsule containing 0.5 g, approximately 1.5 g crude drug.

The dosage of the capsule is 2#, tid. The disaggregating time of this invented capsule is 4-6 minutes after taking orally, finished product have more polysaccharides, higher average yield of Paeonol and ursolic acid and 0.517-0.569% marmelosin; compare to present technique, this invented capsule with following merits: take effect fast, with good curative effect, fewer oral dose, convenient to use and the quality is steady.

### EXPERIMENT EXAMPLE 1

1. Experiment Medicine

The invented capsule was provided by Kangyuan pharmacy co. Ltd in Lianyungang. Batch No. 970712. Make the invented capsule to suspension with distilled water separately have 9 g, 18 g, 56 g and 112 g crude drugs in 100 ml physic liquor.

2. Experiment Materials:

2.1 Experiment medicine: Same as above.

2.2 Positive Medicine

2.2.1 Yelaixiang Maikang: Produced by Norman Bethune medical university drug manufactory. Batch No. 960814.

2.2.2 Phenformin Hydrochloride Tablets: Produced by Jiangsu jintan drug manufactory. Batch No. 961004.

2.2.3 Guilingji: Produced by Shanxi Chinese medicine factory. Batch No. 970302.

2.2.4 Huangqijing oral liquid: Produced by the drug manufactory of Nanjing Chinese Medicine College.

2.3 Prepare Method:

2.3.1 Make the invented capsule to suspension with distilled water separately have 9 g, 18 g, 56 g and 112 g crude drugs in 100 ml physic liquor.

2.3.2 Make Yelaixiang Maikang to 4%, 54% suspension with distilled water.

2.3.3 Make Phenformin to 0.5% suspension with distilled water.

2.3.4 Make Guilingji to 1.2% suspension with distilled water.

2.3.5 Make Huangqijng to 27% suspension with distilled water.

2.4 Group and Dosage:

2.4.1 Dosage to mouse: Administration volume is 20 ml/kg
(1) Model group, Normal control group: Distilled water
(2) Positive medicine group: Drug solution of positive medicine
(3) High dose group: 18% suspension of the invented capsule (3.6 g/kg)
(4) Low dose group: 9% suspension of the invented capsule (1.8 g/kg)
(The dosage to adult of this invented capsule is about 0.18 g/kg crude drug).

2.4.2 Dosage to rat: Administration volume is 10 ml/kg
(1) Model group, Normal control group: Distilled water
(2) Positive medicine group: Drug solution of positive medicine
(3) High dose group: 18% suspension of the invented capsule (1.8 g/kg)
(4) Low dose group: 9% suspension of the invented capsule (0.9 g/kg).

2.4.3 Dosage to rabbit: Administration volume is 1 ml/kg
(1) Model group: Distilled water
(2) Positive medicine group: Drug solution of positive medicine
(3) High dose group: 112% suspension of the invented capsule (1.12 g/kg)
(4) Low dose group: 56% suspension of the invented capsule (0.56 g/kg).

2.5 Animal Subject

2.5.1 Healthy SD rats, provided by experimental anima center of Nanjing University of traditional Chinese medicine.

2.5.2 Healthy ICR rats, provided by experimental anima center of Nanjing University of traditional Chinese medicine.

2.5.3 Cyan-blue rabbits, provided by experimental anima center of Nanjing railway medical college.

2.5.4 Certificate of the experimental animal environment and facilities: Jiangsu experimental animal environment certificate No. 95102 and 97003.

Certificate of the experimental animal: Jiangsu experimental animal quality certificate No. 97003.

2.6 Reagent

2.6.1 Evaluate kit of Total cholesterol (TCH): Produced by Kexin biotechnology research center. Shanghai. Batch No. 970801, 9804010.

2.6.2 Evaluate kit of High density lipoprotein cholesterol (HDL-C): Produced by Kexin biotechnology research center. Shanghai. Batch No. 970801, 980201.

2.6.3 Evaluate kit of Low density lipoprotein cholesterol (LDL-C): Produced by Kexin biotechnology research center. Shanghai. Batch No. 970801, 980201.

2.6.4 Evaluate kit of Triglyceride (TG): Produced by Kexin biotechnology research center. Shanghai. Batch No. 971202, 980501.

2.6.5 Evaluate kit of Glucose (GLU): Produced by Kexin biotechnology research center. Shanghai. Batch No. 971001.

2.6.6 Evaluate kit of Superoxide dismutase (SOD): Produced by Jiancheng biotechnology research center. Nanjing. Batch No. 980223.

2.6.7 Evaluate kit of malonaldehyde (MDA): Produced by Jiancheng biotechnology research center. Nanjing. Batch No. 980223.

3. Experiment Method and Results:

3.1 The Impact to the Blood Fat Value of Experimental Hyperlipoidemia Rabbit:

3.1.1 Experiment Procedure:

28 male cyan-blue rabbit, weighing 1.9˜2.2 kg, were randomly divided into 4 groups: Model group, Positive medicine Yelaixiang Maikang group, High and low dose of invented capsule group. All rabbits were raised with high-grease bait vessel (1% cholesterol, 10% lard, 15% egg yolk, 74% common bait vessel), feeding continuously 4 weeks to form hyperlipoidemia. At the same time take orally certain experimental medicine 1 time each day. Take another 7 rabbits as normal control group. All rabbits were fasting diet 24 hours after last administration, then taking blood from vein of ear edge, separating blood serum, using evaluate kit evaluated triglyceride (TG), total cholesterol (TCH), low density lipoprotein cholesterol (LDL-C), high density lipoprotein cholesterol (HDL-C), figure out TC/HDL-C and Atherosclerotic index (A1), then using t-value method to compare the significance with model group.

Atherosclerotic index (A1): (TC—HDL-C)/HDL-C.

3.1.2 Experimental Result:

The invented soft capsule can obviously reduce TG, TCH, LDL-C, TCH/HDL-C ratio and AI of experimental rabbits with hyperlipoidemia, there is remarkable significance compare to model group, see table 1.

3.2 The Impact to the Blood Fat Value of Experimental Hyperlipoidemia Rat:

3.2.1 Experiment Procedure:

60 male SD rat, weighting 200˜250 g, were raised with high-grease bait vessel (2% cholesterol, 10% lard, 0.2% thyreostat, 87.8% common bait vessel), feeding continuously 4 weeks, then taking blood from eye sockets, using evaluate kit evaluated total cholesterol (TCH), select 40 rats with hyperlipemia whose TCH value is 7˜11 mmol/L, Which were randomly divided into 4 groups: Model group, Positive medicine Yelaixiang Maikang group, High and low dose of invented capsule group. All rats were intragastric administration 1 time each day in 4 weeks. Take another 10 rats as normal control group. All rats were fasting diet 16 hours after last administration, then taking blood from eye sockets, separating blood serum, using evaluate kit evaluated triglyceride (TG), total cholesterol (TCH), low density lipoprotein cholesterol (LDL-C), high density lipoprotein cholesterol (HDL-C), figure out TC/HDL-C and Atherosclerotic index (A1), then using t-value method to compare the significance with model group.

3.2.2 Experimental Result:

The invented soft capsule can obviously reduce TG, TCH, LDL-C, TCH/HDL-C ratio and AI of experimental rats with hyperlipoidemia, there is remarkable significance compare to model group, see table 2.

3.3 The Impact to the Blood Glucose Value of Experimental Mouse With Hyperglycaemia

3.3.1 Experiment Procedure:

100 male mice, weighting 25˜30 g, were given Streptozocin on the dosage of 180 mg/kg by intraperitoneal injection. taking blood from eye sockets of the survivals 72 hours later, using evaluate kit evaluated serum glucose, select 40 mice with hyperglycosemia whose serum glucose value is 18˜33 mmol/L, Which were randomly divided into 4 groups: Model group, Positive medicine Phenformin group, High and low dose of invented capsule group. All mice were intragastric administration 1 time each day in 10 days; Take another 10 mice as normal control group. All mice were fasting diet 12 hours after last administration, then taking blood from eye sockets, separating blood serum, using evaluate kit evaluated serum glucose value with Glucose enzymic method, then using t-value method to compare the significance with model group.

3.3.2 Experimental Result:

The invented soft capsule can obviously reduce serum glucose of the experimental mice with hyperglycaemia lead by Streptozocin, there is remarkable significance compare to model group, see table 3.

3.4 The Impact to the Blood Glucose Value of Experimental Rats With Hyperglycaemia

3.4.1 Experiment Procedure:

160 male rats, weighting 200˜250 g, were given alloxan on the dosage of 200 mg/kg by intraperitoneal injection. taking blood from eye sockets of the survivals 72 hours later, using evaluate kit evaluated serum glucose, select 40 rats with hyperglycosemia whose serum glucose value is 20˜25 mmol/L, Which were randomly divided into 4 groups: Model group, Positive medicine Phenformin group, High and low dose of invented capsule group. All rats were intragastric administration 1 time each day in 10 days; Take another 10 mice as normal control group. All mice were fasting diet 12 hours after last administration, then taking blood from eye sockets, separating blood serum, using evaluate kit evaluated serum glucose value with Glucose enzymic method, then using t-value method to compare the significance with model group.

3.4.2 Experimental Result:

The invented soft capsule can obviously reduce serum glucose of the experimental rats with hyperglycaemia lead by alloxan, there is remarkable significance compare to model group, see table 4.

3.5 The Impact to the Content of SOD and LPO in Serum of Experimental Rats

3.5.1 Experiment Procedure:

40 SD rats, half female and half male, weighting 350˜450 g, were randomly divided into 4 groups: Normal control group, Positive medicine Guilingji group, High and low dose of invented capsule group. All rats were intragastric administration 1 time each day in 10 days; taking blood from eye sockets 24 hours after last administration, separating blood serum, using evaluate kit of Superoxide dismutase (SOD), shade selection at 550 nm wavelength with xanthine oxidase to evaluated optical density (OD), then calculate energy of SOD according to formula (Nitrite unit, NU/ml); using evaluate kit of malondialdehyde (MDA), shade selection at 532 nm wavelength with malondialdehyde-thio-malonylurea to evaluated optical density (OD), then calculate the content of MDA in the serum of rats according to formula, then using t-value method to compare the significance with normal control group.
Calculate formula:

$$SOD activity ( NU \text{/} ml ) = \frac{Contrast OD - Evaluated tube OD}{Contrast tube OD} \div 50 \% \times sample diluted times$$

$$MDA ( nM \text{/} ml ) = \frac{EvaluatedtubeOD - Contrast vacant tube OD}{StandardtubeOD - Standerdvacant tube OD} \times 10 nM \text{/} ml \times sample diluted times$$

3.5.2 Experimental Result:

The invented soft capsule can obviously enhance the energy of SOD and reduce the content of MDA in the serum of rats, there is remarkable significance compare to normal control group, see table 5.

3.6 The Impact to the Organ Index Number of Mice's Immune Organ

3.6.1 Experiment Procedure:

40 healthy and young ICR mice, half female and half male, weighting 12˜14 g, were randomly divided into 4 groups: Normal control group, Positive medicine Huangqijing oral liquid group, High and low dose of invented capsule group. All mice were intragastric administration 1 time each day in 10 days; 24 hours after last administration, kill the mice with certebrae colli luxation method, then taking the thoracic gland and spleen, weighing to calculate the index number of thoracic gland and spleen (organ index number=organ weight (mg)/body weight (g), then using t-value method to compare the significance with normal control group.

3.6.2 Experimental Result:

The invented soft capsule can obviously enhance the index number of thoracic gland and spleen, there is remarkable significance compare to normal control group, see table 6.

3.7 The Impact to Inflammatory Dropsy of Delayed Type Hypersensitivity With Mice Skin Lead by DNCB

3.7.1 Experiment Procedure:

50 ICR mice, half female and half male, weighting 20˜22 g. Using 0.02 ml 7% 2,4-dinitro-chlorobenzene (DNCB) acetone solution to lead hypersensitiveness with hypodermical injection in the back of mice. Which were randomly divided into 5 groups: Normal control group, Model group, Positive medicine Guilingji group, High and low dose of invented capsule group. Start administration when hypersensitiveness is formed, All mice were intragastric administration 1 time each day in 10 days; In 4th and 6th day after hypersensitiveness is formed, All mice were given Cyclophosphamide on the dosage of 30 mg/kg with intraperitoneal injection except normal control group. Smearing 1% DNCB Sesame Oil solution 0.03 ml on the right ear of all mice to stir up excitation an hour after last administration, Kill the mice 16 hours later, using round punch (d=8 mm) to get a piece of round ear tissues from both left and right ear, weighting, apply the difference of weight between left and right ear tissues as the standard of inflammatory dropsy degree of delayed type hypersensitivity with mice skin lead by DNCB, then using t-value method to compare the significance with model group.

3.7.2 Experimental Result:

The invented soft capsule can obviously enhance the difference of weight between left and right ear tissues, there is remarkable significance compare to model group, see table 7.

3.8 The Impact to the Content of Serum Hemolysin of Mice

3.8.1 Experiment Procedure:

50 ICR mice, half female and half male, weighting 20˜22 g. were Intraperitoneal injection with 0.21 ml 5% chicken red blood cell and Sodium Chloride suspension to immune. Which were randomly divided into 5 groups: Normal control group, Model group, Positive medicine Guilingji group, High and low dose of invented capsule group. Start administration after immunized, all mice were intragastric administration 1 time each day in 7 days, In 4th and 6th day after hypersensitiveness is formed, All mice were given Cyclophosphamide 30 mg/kg with intraperitoneal injection except normal control group. Taking blood from the eyeball of mice an hour after last administration, centrifugal separating serum and then diluted it with Sodium Chloride to 100 times, taking the diluted serum 1 ml, mixed with 5% chicken red blood cell and Sodium Chloride suspension 0.5 ml, 10% complement 0.5 ml, keeping constant temperature at 37° C. 30 minutes, then stop the reaction in refrigerator at 0° C. Centrifuging it to get supernate, then shade selection at 540 nm wavelength to evaluated optical density (OD), using OD as the standard of serum hemolysin level, then using t-value method to compare the significance with model group.

3.8.2 Experimental Result:

The invented soft capsule can obviously enhance the content of serum hemolysin of mice, there is remarkable significance compare to model group, see table 8.

3.9 The Impact to the Ability of Mice to Endure Oxygen Deficiency With Normal Pressure

3.9.1 Experiment Procedure:

40 ICR mice, half female and half male, weighting 19˜22 g, were randomly divided into 4 groups: Normal control group, Positive medicine Huangqijing oral liquid group, High and low dose of invented capsule group. All mice were intragastric administration 1 time each day in 10 days. An hour after last administration, take all mice into white 125 ml wide-mouthed bottle which have 15 g natrica calx, smearing Vaseline around the bottle cap to seal up, count time immediately, taking respiration ceases as standard, observe the survival time of the mice, then using t-value method to compare the significance with normal control group.

3.9.2 Experimental Result:

The invented soft capsule can obviously extend the experimental mice's endurable time of oxygen deficiency with normal pressure, there is remarkable significance compare to normal control group, see table 9.

3.10 The Impact to Mice's Swimming Time in Low Temperature

3.10.1 Experiment Procedure:

50 female ICR mice, weighting 18˜20 g, were randomly divided into 4 groups: Normal control group, Positive medicine Huangqijing oral liquid group, High and low dose of invented capsule group. All mice were intragastric administration 1 time each day in 7 days; Fasting diet 12 hours before last administration, An hour after last administration, take all mice into the glass water-bath which diameter and height were both 30 cm to carry the low-temperature-swimming experiment, the depth is 20 cm, and the temperature is 10±1° C., the weight loading of mice tail is 5% of body weight. Observe the persistence time of the mice swimming in low temperature, and then using t-value method to compare the significance with normal control group.

3.10.2 Experimental Result:

The invented soft capsule can obviously extend the experimental mice's persistence time swimming in low temperature, there is remarkable significance compare to normal control group, see table 10.

4. Conclusion:

The invented soft capsule can obviously reduce TG, TCH, LDL-C, TCH/HDL-C ratio and AI of experimental rats and rabbits with hyperlipoidemia; Obviously reduce serum glucose of the experimental mice with hyperglycaemia lead by Streptozocin and the experimental rats with hyperglycaemia lead by alloxan; obviously enhance the energy of SOD and reduce the content of MDA in the serum of rats; obviously enhance the index number of thoracic gland and spleen, enhance inflammatory dropsy degree of delayed type hypersensitivity with mice skin lead by DNCB; obviously enhance the content of serum hemolysin of mice; obviously extend the experimental mice's endurable time of oxygen deficiency with normal pressure, All the above proved that the invented soft capsule had the functions of reducing blood fat and glucose, enhancing SOD level, reducing lipid peroxidation, enhancing immune function, anti-fatigue and anti-fatigue functions.

TABLE 1
The impact of the invented soft capsule to the blood fat value of
experimental hyperlipoidemia rabbit (x ± SD)

| | Group | | | | |
|---|---|---|---|---|---|
| | | | Yelaixiang | The invented | The invented |
| | Normal | Model | Maikang | soft capsule | soft capsule |
| Dosage(g/kg) | — | — | 0.45 | 1.12 | 0.56 |
| Animal number | 7 | 7 | 7 | 7 | 7 |
| TG | 16.2 ± 0.68 | 10.55 ± 4.66 | 1.44 ± 0.66*** | 1.70 ± 0.94*** | 2.76 ± 0.27*** |
| (mmol/L) | | | | | |
| TCH | 0.99 ± 0.18 | 44.00 ± 9.01 | 5.00 ± 1.10*** | 1.62 ± 0.45*** | 5.05 ± 2.04*** |
| (mmol/L) | | | | | |
| HDL-C | 0.81 ± 0.18 | 2.09 ± 0.30 | 0.93 ± 0.42*** | 0.79 ± 0.19*** | 0.63 ± 0.18*** |
| (mmol/L) | | | | | |
| LDL-C | 0.54 ± 0.18 | 7.38 ± 0.25 | 3.05 ± 1.63*** | 1.64 ± 0.49***Δ | 3.41 ± 0.76*** |
| (mmol/L) | | | | | |
| TCH/ | 1.30 ± 0.41 | 21.48 ± 5.36 | 5.16 ± 1.95*** | 2.15 ± 0.80*** | 7.87 ± 3.43*** |
| HDL-C | | | | | |
| AI | 0.30 ± 0.41 | 20.48 ± 5.36 | 4.16 ± 1.95*** | 1.15 ± 0.80*** | 6.87 ± 3.43*** |

Compare to model group: ***P < 0.001; ΔP < 0.05.

TABLE 2
The impact of the invented soft capsule to the blood fat value of experimental hyperlipoidemia rats (x ± SD)

| | | group | | | | |
|---|---|---|---|---|---|---|
| | | Normal | Model | Yelaixiang | The invented | The invented |
| | | control | group | Maikang | soft capsule | soft capsule |
| Dosage(g/kg) | | — | — | 0.4 | 1.8 | 0.9 |
| Animal | | 10 | 10 | 10 | 10 | 10 |
| number | | | | | | |
| TG | Before | 1.53 ± 0.16 | 8.92 ± 1.12 | 8.93 ± 1.04 | 8.91 ± 1.12 | 8.89 ± 1.13 |
| | administration | | | | | |
| (mmol/L) | After | 1.58 ± 0.13 | 11.50 ± 2.06 | 3.18 ± 0.65*** | 3.03 ± 0.65*** | 3.48 ± 0.70*** |
| | administration | | | | | |
| TCH | Before | 0.71 ± 0.17 | 2.21 ± 0.34 | 2.11 ± 0.33 | 2.15 ± 0.32 | 2.11 ± 0.37 |
| | administration | | | | | |
| (mmol/L) | After | 0.69 ± 0.18 | 2.86 ± 0.72 | 1.03 ± 0.39*** | 1.15 ± 0.39*** | 1.17 ± 0.35*** |
| | administration | | | | | |
| HDL-C | Before | 0.91 ± 0.22 | 3.38 ± 0.85 | 3.58 ± 0.84 | 3.54 ± 1.15 | 3.57 ± 1.03 |
| | administration | | | | | |
| (mmol/L) | After | 0.95 ± 0.26 | 4.13 ± 0.81 | 1.84 ± 0.68*** | 2.02 ± 0.59*** | 1.97 ± 0.77*** |
| | administration | | | | | |
| LDL-C | Before | 0.80 ± 0.20 | 6.49 ± 1.17 | 6.60 ± 1.13 | 6.31 ± 1.10 | 6.09 ± 1.05 |
| | administration | | | | | |
| (mmol/L) | After | 0.76 ± 0.14 | 7.75 ± 1.68 | 1.75 ± 0.47*** | 1.56 ± 0.66***Δ | 2.02 ± 0.44*** |
| | administration | | | | | |
| TCH/ | Before | 1.77 ± 0.45 | 2.74 ± 0.58 | 2.55 ± 0.29 | 2.77 ± 0.91 | 2.63 ± 0.59 |
| | administration | | | | | |
| HDL-C | After | 1.79 ± 0.55 | 2.80 ± 0.24 | 1.82 ± 0.33*** | 1.56 ± 0.32*** | 2.01 ± 0.75** |
| | administration | | | | | |
| AI | Before | 0.77 ± 0.45 | 1.74 ± 0.58 | 1.55 ± 0.29 | 1.77 ± 0.91 | 1.63 ± 0.59 |
| | administration | | | | | |
| | After | 0.79 ± 0.55 | 1.80 ± 0.24 | 0.82 ± 0.33*** | 0.56 ± 0.32*** | 1.01 ± 0.75** |
| | administration | | | | | |

Compare to Model group: **P < 0.01, ***P < 0.001; ΔP < 0.

TABLE 3
The impact of the invented soft capsule to the blood glucose value
of experimental mice with hyperglycaemia (x ± SD)

| | | | GLU | (mmol/L) |
|---|---|---|---|---|
| | Dosage | Animal | Before | After |
| group | (g/kg) | number | administration | administration |
| Normal control | — | 10 | 5.59 ± 0.71 | 5.27 ± 0.93 |
| Model group | — | 10 | 25.94 ± 4.74 | 39.72 ± 8.59 |
| Phenformin | 0.1 | 10 | 25.43 ± 3.93 | 12.51 ± 6.93*** |
| The invented | 3.6 | 10 | 25.27 ± 4.07 | 11.72 ± 4.62*** |
| soft capsule | | | | |
| The invented | 1.8 | 10 | 25.26 ± 4.07 | 23.65 ± 4.49*** |
| soft capsule | | | | |

Compare to Model group: ***P < 0.001.

TABLE 4
The impact of the invented soft capsule to the blood glucose value
of experimental rats with hyperglycaemia (x ± SD)

| | | | GLU | (mmol/L) |
|---|---|---|---|---|
| | Dosage | Animal | Before | After |
| group | (g/kg) | number | administration | administration |
| Normal control | — | 10 | 5.95 ± 0.62 | 6.36 ± 0.81 |
| Model group | — | 10 | 22.71 ± 1.53 | 24.77 ± 4.31 |
| Phenformin | 0.1 | 10 | 22.72 ± 1.53 | 14.52 ± 4.99*** |
| The invented | 1.8 | 10 | 22.65 ± 1.52 | 12.58 ± 4.97*** |
| soft capsule | | | | |
| The invented | 0.9 | 10 | 22.68 ± 1.55 | 17.87 ± 5.54** |
| soft capsule | | | | |

Compare to Model group: **P < 0.01, ***P < 0.001

[symbol]

TABLE 5
The impact of the invented soft capsule to the content of SOD and
LPO in serum of experimental rats (x ± SD)

| | | | SOD | |
|---|---|---|---|---|
| | Dosage | Animal | energy | MDA |
| group | (g/kg) | number | (NU/ml) | (nM/ml) |
| Normal control | — | 10 | 583.2 ± 175.6 | 167.1 ± 29.3 |
| Group | | | | |
| Guilingji | 0.12 | 10 | 771.3 ± 182.8* | 135.6 ± 27.8* |
| The invented | 1.80 | 10 | 873.7 ± 211.1** | 121.0 ± 28.7** |
| soft capsule | | | | |
| The invented | 0.90 | 10 | 750.3 ± 206.0 | 137.8 ± 28.0* |
| soft capsule | | | | |

Compare to Model group: *P < 0.05, **P < 0.01

TABLE 6
The impact of the invented soft capsule to the index number
of thoracic gland and spleen (x ± SD)

| | | | | index number | index number |
|---|---|---|---|---|---|
| | Dosage | Animal | Weight | of thoracic | of spleen |
| group | (g/kg) | number | (g) | gland(mg/g) | (mg/g) |
| Normal control | — | 10 | 21.25 ± 3.40 | 3.04 ± 0.66 | 4.61 ± 0.47 |
| Huangqijing oral liquid | 5.4 | 10 | 24.30 ± 3.32 | 5.00 ± 1.00** | 5.68 ± 1.38* |
| The invented soft capsule | 3.6 | 10 | 19.34 ± 1.72 | 4.05 ± 0.93*Δ | 5.66 ± 1.30*ΔΔ |
| The invented soft capsule | 1.8 | 10 | 19.92 ± 2.81 | 3.84 ± 0.68* | 5.08 ± 1.15 |

Compare to Model group: *P < 0.05, **P < 0.01; ΔP < 0.05; ΔΔP < 0.01

[symbol]

TABLE 7
The impact of the invented soft capsule to inflammatory dropsy of delayed type
hypersensitivity with mice skin lead by DNCB (x ± SD)

| | | | | | the difference between |
|---|---|---|---|---|---|
| | Dosage | Animal | Weight of tissues | Weight of tissues | left and right ear |
| group | (g/kg) | number | from the left ear(mg) | from the right ear(mg) | tissues(mg) |
| Normal control | — | 10 | 13.40 ± 0.52 | 15.00 ± 1.25 | 16.0 ± 0.97 |
| Model group | — | 10 | 14.00 ± 1.41 | 14.60 ± 1.50 | 0.60 ± 1.58 |
| Guilingji | 0.24 | 10 | 12.70 ± 1.16 | 17.90 ± 2.73 | 5.20 ± 2.82*** |
| The invented | 3.6 | 10 | 13.60 ± 1.70 | 20.70 ± 4.06 | 7.10 ± 4.01***ΔΔ |
| soft capsule | | | | | |
| The invented | 1.8 | 10 | 13.30 ± 1.83 | 16.00 ± 1.41 | 2.70 ± 1.64** |
| soft capsule | | | | | |

Compare to Model group: **P < 0.01, ***P < 0.001; ΔΔP < 0.01.

TABLE 8
The impact of the invented soft capsule to the content
of serum hemolysin of mice (x ± SD)

| | | | optical |
|---|---|---|---|
| | Dosage | Animal | density |
| group | (g/kg) | number | (OD) |
| Normal control | — | 10 | 0.192 ± 0.099 |
| Model group | — | 10 | 0.137 ± 0.006 |
| Guilingji | 0.24 | 10 | 0.170 ± 0.008*** |
| The invented soft capsule | 3.6 | 10 | 0.251 ± 0.099***ΔΔ |
| The invented soft capsule | 1.8 | 10 | 0.171 ± 0.028** |

Compare to Model group: **P < 0.01, ***P < 0.001, ΔΔP < 0.01.

TABLE 9
The impact of the invented soft capsule to the ability of mice to endure
oxygen deficiency with normal pressure (x ± SD)

| | Dosage | Animal | Survival |
|---|---|---|---|
| group | (g/kg) | number | time(min) |
| Normal control | — | 10 | 16.39 ± 1.71 |
| Huangqijing oral liquid | 5.4 | 10 | 16.30 ± 1.68 |
| The invented soft capsule | 3.6 | 10 | 20.51 ± 4.42**ΔΔ |
| The invented soft capsule | 1.8 | 10 | 18.23 ± 2.20 |

Compare to Model group: **P < 0.01, ΔΔP < 0.

TABLE 10
The impact of the invented soft capsule to mice's swimming
time in low temperature (x ± SD)

| | Dosage | Animal | swimming time in low |
|---|---|---|---|
| group | (g/kg) | number | temperature(min) |
| Normal control | — | 10 | 3.14 ± 0.89 |
| Huangqijing oral liquid | 5.4 | 10 | 5.62 ± 1.59** |
| The invented soft | 3.6 | 10 | 6.29 ± 2.33** |
| capsule | | | |
| The invented soft | 1.8 | 10 | 4.23 ± 1.04 |
| capsule | | | |

Compare to Model group: **P < 0.01

### EXPERIMENT EXAMPLE 2

### Clinical Trial

I The Criteria to Chose the Cases

(I) The Diagnostic Criteria

1. The diagnostic criteria of the deficiency of kidney yin (referring the diagnostic criteria of the deficiency syndromes established in the 3rd nation integrated traditional and western medicine conference): aching pain in the waist and knees, burning sensation of five centers, dry mouth and throat, dizziness, ear ringing or deafness, night sweat, constipation, red tongue body and thready and rapid pulse.

2 The Diagnostic Criteria of the Hyperlipemia:

On the normal diet, the serum TC≧6.0 mmol/L, or the TG≧1.54 mmol/L for 2 times within 2 weeks; the HDL≦1.04 mmol/L in the male or HDL≦1.17 mmol/L in the female can be diagnosed as hyperlipemia too.

3 The Diagnostic Criteria of the Diabetes II: (Referring to the Corresponding Criteria Made by WHO at 1980)
(1) There are the typical symptoms of diabetes and the blood sugar any time is more than 11.1 mmol/L, or the fasting blood sugar without meal is higher than 7.8 mmol/L.
(2) There are the typical symptoms; though the blood sugar is lower than the level above, the OGTT (method of 75 g glucose) indicates that the blood sugar 2 hours after the sugar intake is higher than 11.1 mmol/L.
(3) If there are not symptoms related to the diabetes, the criteria above must be supplied with another criterion that in the OGTT, the blood sugar 1 hour after the sugar intake must higher than 11.1 mmol/L, or the blood sugar 2 hours after the sugar intake must higher than 11.1 mmol/L in another OGTT, or another fasting blood sugar before the meal is higher than 7.8 mmol/L.

According to the criteria above, the patients can be diagnosed as diabetes II.

(II) The Criteria About the Cases in the Clinical Trial

1. The Warranty of the Disease Type Selection

Based on the experimental results that the invented soft capsule could lower the blood sugar and blood fat and the clinical function that the invented medicine could improve the symptoms of emaciation-thirst disease, the diabetes II and hyperlipemia patients who were diagnosed as deficiency of kidney yin were brought into the clinical trial. Some selected patients were brought into the open treatment group (the diseases were not limited).

2. The Selection Criteria
(1) The patients had the symptoms related to deficiency of kidney yin, and the corresponding score was higher than 10.
(2) The patients of hyperlipemia or diabetes II or the people who had been diagnosed before but had controlled the condition stably by the diet or drugs were brought into the trial. The patients who were suffering from the other diseases but diagnosed to be the syndrome of deficiency of kidney yin were brought into the open treatment group.
(3) The ages of the cases varied from 18 to 70 years old.
(4) All the patients were not forced.

3 The Elimination Criteria
(1) The patients were suffering from the other serious diseases in the cardiovascular system, cerebrovascular system, liver, kidney, hemopoietic system and spirit.
(2) The cases' age was beyond the range 18-70 years old.
(3) The women were in the period of pregnancy and lactation. The patients were allergic to this medicine.
(4) The patients whose scores of the deficiency of kidney yin were lower than 10 or the patients who had the combined symptoms of the deficiency of kidney yin and the damp heat such like the thick-greasy fur should be eliminated.
(5) Any patient who was not according to the selection criteria or didn't take the medicine on the prescription so that the curative effect couldn't be evaluated, or was short of the important data so that the curative effect and security could not be appraised would be eliminated.

II The Grouping and Administrating Method

1. Comparison Method:

The auto control before and after the treatment was adopted. There were 100 patients in the treatment group I who received the treating of the invented medicine, while 30 patients in the treatment group II who received the treating of any usual medicine except the invented medicine.

2. The Method of Administration

The single blind method was adopted and the patients in treatment group I were treated with the invented soft capsule on the dose of 2 capsules (each capsule equals to 1.5 g crude drug) one time, 3 times a day.

3. The period of treatment: 6 weeks.

4. The source of the cases: the outpatients and the inpatients were not limited. At the end of the trial, 152 outpatients and 78 inpatients had attended the trial and the ratio of the inpatients was 33.91%. The outpatients were urged to take the medicine rigidly and every week there were special persons who were in charge of the follow-up survey.

III The Indexes of Observation

1. The Indexes Related to the Curative Effect
(1) The score change of the symptoms and physical signs related to the deficiency of the kidney yin.
(2) The change of the blood fat and sugar of the hyperlipemia patients and type II diabetes patients respectively.

2. The Indexes Related to the Security
(1) The general condition of the patients.
(2) The 3 routine tests of blood, urine and stool.
(3) The function tests of liver and kidney. (SGPT, A/G, BUN, Scr)
(4) ECG

3. The Method of Observation
(1) Inquiring and recording the related things about the administration.
(2) Evaluating and recording the symptoms related to the deficiency of the kidney yin before the treatment and 2 weeks, 4 weeks and 6 weeks after the treatment. Appraising the change of the blood sugar and blood fat and evaluating the indexes of security. Recording the results above.

IV The Criteria About the Curative Effect

(I) The Criteria of Evaluation About the Deficiency of the Kidney Yin

Four grades: clinical recovery, significantly effective, effective and ineffective.
1. Clinical recovery: the symptoms, physical signs or the score of the deficiency of the kidney yin decreased by 90%.
2. Significantly effective: the symptoms, physical signs or the score of the deficiency of the kidney yin decreased by 60-90%.
3. Effective: the symptoms, physical signs or the score of the deficiency of the kidney yin decreased by 30-60%.
4. Ineffective: the symptoms, physical signs didn't improve or the score of the deficiency of the kidney yin decreased less than 30%.

(II) The Criteria of Evaluation About the Curative Effect on the Diabetes. (Referring to the <The new Drugs' Clinical Research Governing Principles of the TCM> Fascicule 1, Page 216)

1. Significantly effective: the symptoms are almost disappeared after the treating and the fasting blood sugar (before the breakfast) is lower than 7.2 mmol/L (130 mg/dl), the postprandial blood sugan (2 hours after the meal) is lower than <8.3 mmol/L (150 mg/dl); the urine sugar quantity is less than 10.0 g/24 hours; or the level of blood sugar and the 24 hours urine sugar quantity decreased by 30% after the treatment.

2. Effective: the symptoms improves significantly after the treatment; the fasting blood sugar (before the breakfast) is lower than 8.3 mmol/L (150 mg/dl), the postprandial blood sugar (2 hours after the meal) is lower than <10 mmol/L (180 mg/dl); the urine sugar quantity is less than 25.0 g/24 hours; or the level of blood sugar and the 24 hours urine sugar quantity decreased by 10% after the treatment.

3. Ineffecitive: the improvement of the blood sugar and urine sugar doesn't reach the level of effective.

(III) The Criteria of Evaluation About the Curative Effect on the Hyperlipemia. (Referring to the <The New Drugs' Clinical Research Governing Principles of the TCM> Fascicule 2, Page 172)

4. Clinical recovery: the symptoms and physical signs disappeared and the related indexes of laboratory examination turned normal after the treatment.

5. Significantly effective: the symptoms are almost disappeared after the treating and one of the indexes has reached the criterion as following: TC decreased≧20%, TG decreased≧40%, HDL-C increased≧0.26 mmol/L (10 mg/dl), TC-HDL-C/HDL-C decreased≧20%.

6. Effective: one of the indexes has reached the criterion as following: TC decreased by 10%-20%, TG decreased by 20%-40%, HDL-C increased by 0.104 mmol/L (4 mg/dl)-0.26 mmol/L (10 mg/dl), TC-HDL-C/HDL-C decreased by 10%-20%.

7. Ineffective: the symptoms, physical signs and the indexes of laboratory examination didn't improve clearly.

V. The Untoward Reactions and the Side-Effects

Observing and recording all the untoward reactions and the side-effects in the period of treating.

VI Results:

(I) The Results of the Clinical Trials About the Hyperlipemia and Diabetes

TABLE 11
The results of the hyperlipemia treating trial

| | | | | | | | Ratio of clinical | |
|---|---|---|---|---|---|---|---|---|
| | | | Curative effect | | | | recovery and | Total ratio of |
| | | | Clinical | Significantly | | | significantly | the effective |
| Disease | Group | Number | recovery | effective | effective | ineffective | effective (%) | (%) |
| Hyperlipermia | Treatment | 50 | 5 | 19 | 19 | 7 | 48.0 | 86.0 |
| | group | | | | | | | |

Based on the <The new drugs' clinical research governing principles the TCM on the hyperlipemia>, the ratio of clinical recovery and significantly effective is 48% and the total effective ratio is 86%.

TABLE 12
The results of the diabetes treating trial

| | | | | | | Ratio of clinical | |
|---|---|---|---|---|---|---|---|
| | | | Curative effect | | | recovery and | Total ratio of |
| | | | Significantly | | | significantly | the effective |
| Disease | Group | Number | effective | effective | ineffective | effective (%) | (%) |
| Diabetes | Treatment | 50 | 9 | 25 | 16 | 18.0 | 68.0 |
| | group | | | | | | |

Based on the <The new drugs' clinical research governing principles of the TCM on the diabetes>, the ratio of significantly effective is 18.0% and the total effective ratio is 68.0%.

(I) The Change of the Blood Fat and the Blood Sugar Before and After the Treatment

TABLE 13
The decrease of the blood fat after the treatment (mmol/L)

| | | | Before the treatment | | | After the treatment | | | Comparison intergroup | | Comparison among groups | |
|---|---|---|---|---|---|---|---|---|---|---|---|---|
| Disease | Group | Number | \overline{x} ± SD | T | p | \overline{x} ± SD | t | p | t | p | t | p |
| TC | Treatment group | 50 | 5.84 ± 1.66 | 0.62 | >0.05 | 4.96 ± 1.41 | 0.42 | >0.05 | 5.49 | <0.01 | 0.36 | >0.05 |
| TG | Treatment group | 50 | 3.64 ± 2.11 | 0.22 | >0.05 | 2.78 ± 1.61 | 0.28 | >0.05 | 4.62 | <0.01 | 1.00 | >0.05 |
| HDL | Treatment group | 50 | 1.70 ± 0.90 | 0.62 | >0.05 | 1.62 ± 0.59 | 0.53 | >0.05 | 0.86 | >0.05 | 1.28 | >0.05 |

TABLE 14
The decrease of the blood sugar after the treatment (mmol/L)

| | | | | | | | | | | | Comparison | |
|---|---|---|---|---|---|---|---|---|---|---|---|---|
| | | | | | | | | | Comparison | | among | |
| | | | Before the treatment | | | After the treatment | | | intergroup | | groups | |
| Disease | Group | Number | \overline{x} ± SD | t | p | \overline{x} ± SD | t | p | t | p | t | P |
| fasting blood sugar | Treatment | 50 | 6.67 ± 1.39 | 1.81 | >0.05 | 6.23 ± 1.20 | 1.88 | >0.05 | 3.00 | <0.01 | 0.06 | >0.05 |
| | group | | | | | | | | | | | |
| Postprandial blood | Treatment | 48 | 11.05 ± 3.13 | 0.15 | >0.05 | 9.71 ± 2.38 | 0.16 | >0.05 | 3.93 | <0.01 | 0.39 | >0.05 |
| sugar | group | | | | | | | | | | | |

According to table 13 and table 14, the invented medicine could lower the TG, TC in the hyperlipemia patients. The decrease of the blood fat after the treatment was significant; in the diabetes patients, the medicine could lower the blood sugar before and after meal; the decrease of the blood sugar was significant.

### PRACTICE EXAMPLE 1

| Prepared rehmannia root | 960 | g |
|---|---|---|
| root-bark of peony | 360 | g |
| Indian bread | 360 | g |
| Refined edible vegetable oil | Right amount | |
| Prepared dogwood fruit | 480 | g |
| chinese yam | 480 | g |
| alisma rhizome | 360 | g |

The root-bark of peony is soaked in the water 12 times of the herb for 3 hours, and then the root-bark of peony is distilled by the water vapour for 12 hours. Collecting the distilled liquor until it reaches the 8 times weight of the herb and preserving the mother liquor separately. Cooling the distilled liquor at 0-4° C. for 12 hours and filtrating to collect the paeonol; dehydrating the wet paeonol by the cold wind and preserving the dry paeonol in airtight condition. The solid residual is mixed with the prepared rehmannia root, Chinese yam and Indian bread. The mixture is decocted in the water 8 times weight of the herbs for 3 times. Each time continues 1.5 hours. The 3 piece of decocted liquor are mixed and added into the paeonol mother liquor. Concentrating the mixture to a extracturm with the relative density 1.10 at 80° C., centrifugating and concentrating the extracturm to extracturm I with the relative density 1.13 at 80° C. The dogwood fruit and the alisma rhizome are crushed to coarse powder and added 8 times weight of 60% alcohol to extract refluxingly for 3 times. Each time is 3 hours. Collecting the filtrated liquors and retrieving the alcohol, and then concentrating the filtrated liquor to the extracturm II with the relative density of 1.14 at 80° C.; mixing the extracturm I and II, then concentrating the mixture to a thick paste with the relative density of 1.30-1.35; dehydrating the thick paste in the vacuum, crushing the dry medicine to powder and then sifting the powder through the screen. Crushing the paeonol and mixing it with the sifted extracturm powder, adding right amount of supplementary materials and milling the mixture with the colloid mill; sifting the milled powder and putting it into 1000 soft capsules. Each soft capsule is 1.0 g weight and equals to 3.0 g crude drug.

### PRACTICE EXAMPLE 2

| Prepared rehmannia root | 480 | g |
|---|---|---|
| root-bark of peony | 180 | g |
| Indian bread | 180 | g |
| Refined edible vegetable oil | Right amount | |
| Prepared dogwood fruit | 240 | g |
| chinese yam | 240 | g |
| alisma rhizome | 180 | g |

The root-bark of peony is soaked in the water 10 times of the herb for 2 hours, and then the root-bark of peony is distilled by the water vapor for 10 hours. Collecting the distilled liquor until it reaches the 8 times weight of the herb and preserving the mother liquor separately. Cooling the distilled liquor at 0-4° C. for 12 hours and filtrating to collect the paeonol; dehydrating the wet paeonol by the cold wind and preserving the dry paeonol in airtight condition. The solid residual is mixed with the prepared rehmannia root, Chinese yam and Indian bread. The mixture is decocted in the water 6 times weight of the herbs for 2 times. Each time continues 1 hours. The 2 piece of decocted liquor are mixed and added into the paeonol mother liquor. Concentrating the mixture to a extracturm with the relative density 1.10 at 80° C., centrifugating and concentrating the extracturm to extracturm I with the relative density 1.13 at 80° C. The dogwood fruit and the alisma rhizome are crushed to coarse powder and added 6 times weight of 70% alcohol to extract refluxingly for 2 times. Each time is 4 hours. Collecting the filtrated liquors and retrieving the alcohol, and then concentrating the filtrated liquor to the extracturm II with the relative density of 1.14 at 80° C.; mixing the extracturm I and II, then concentrating the mixture to a thick paste with the relative density of 1.30-1.35; dehydrating the thick paste in the vacuum, crushing the dry medicine to powder and then sifting the powder through the screen. Crushing the paeonol and mixing it with the sifted extracturm powder, adding right amount of supplementary materials and milling the mixture with the colloid mill; sifting the milled powder and putting it into 1000 soft capsules. Each soft capsule is 0.5 g weight and equals to 1.5 g crude drug.

1. A composition of medicine made from the original herbs as following:

| | |
|---|---|
| Prepared Rehmannia Root | 300-1000 part-by-weight |
| Prepared Asiatic Cornelian Cherry Fruit | 50-600 part-by-weight |
| Tree peony Bark | 100-450 part-by-weight |
| Common Yan Rhizome | 150-600 part-by-weight |
| Indian Bread | 100-450 part-by-weight |
| Oriental Waterplantain Rhizome | 100-450 part-by-weight. |

2. The composition of medicine according to claim 1 made from the original herbs as following:

| | |
|---|---|
| Prepared Rehmannia Root | 960 part-by-weight |
| Prepared Asiatic Cornelian Cherry Fruit | 480 part-by-weight |
| Tree peony Bark | 360 part-by-weight |
| Common Yan Rhizome | 480 part-by-weight |
| Indian Bread | 360 part-by-weight |
| Oriental Waterplantain Rhizome | 360 part-by-weight. |

3. The composition of medicine according to claim 1 made from the original herbs as following:

| | |
|---|---|
| Prepared Rehmannia Root | 480 part-by-weight |
| Prepared Asiatic Cornelian Cherry Fruit | 240 part-by-weight |
| Tree peony Bark | 180 part-by-weight |
| Common Yan Rhizome | 240 part-by-weight |
| Indian Bread | 180 part-by-weight |
| Oriental Waterplantain Rhizome | 180 part-by-weight. |

4. A soft capsule of medicine made by the method comprising:

| | |
|---|---|
| Prepared Rehmannia Root | 300-1000 part-by-weight |
| Prepared Asiatic Cornelian Cherry Fruit | 150-600 part-by-weight |
| Tree peony Bark | 100-450 part-by-weight |
| Common Yan Rhizome | 150-600 part-by-weight |
| Indian Bread | 100-450 part-by-weight |
| Oriental Waterplantain Rhizome | 100-450 part-by-weight |
| Refined edible vegetable oil | sufficient quantum, |

moistening the tree peony Bark 1-3 hours with 6-12 times water, distill extracting it 6-12 hours with vapor, then collecting distillate 8 times of the Tree peony Bark, keeping mother liquid alone; congealing the distillate 10-14 hours at 0-4° C., leaching paeonol, drying it with cold wind, airtight preserve and keeping alone; Mixing above reside, Prepared Rehmannia Root, Common Yan Rhizome and Indian bread, adding 6-10 times water, then cooking the mixture 1-4 times, each time 0.5-2 hours, combining all the cooked water and the mother liquid of paeonol together, then concentrate it to the extractum with d=1.10 at 80° C., centrifugating with centrifugal separator on the speed of 20000 r/m for 20 minutes, Discard the residue after centrifugated then condensing to the extractum I with relative density is 1.13; Crushing Asiatic Cornelian Cherry Fruit and Oriental Waterplantain Rhizome to coarse powder, adding 50-80% alcohol 4-8 times of it, then extracted it 1-3 times, each time is 2-4 hours, combining the filtrate, retrieving the alcohol, condensing to extractum II with relative density is 1.14 at 80° C.; combining extractum I and extractum II, condensing to thick paste with relative density is 1.30-1.35 at 80° C.; then vacuum drying, crushing and sieving; Crushing Paeonol and mingling with the powdered extract, adding proper amount supplementary materials, then grinding with colloid mill, sieving, pressing to get elastic capsule.

5. The soft capsule of medicine according to claim 4, wherein the method comprising:

| | |
|---|---|
| Prepared Rehmannia Root | 960 part-by-weight |
| Prepared Asiatic Cornelian Cherry Fruit | 480 part-by-weight |
| Tree peony Bark | 360 part-by-weight |
| Common Yan Rhizome | 480 part-by-weight |
| Indian Bread | 360 part-by-weight |
| Oriental Waterplantain Rhizome | 360 part-by-weight |
| Refined edible vegetable oil | sufficient quantum, |

Soaking The root-bark of peony in the water 12 times of the herb for 3 hours, and then distilling the root-bark of peony by the water vapour for 12 hours; Collecting the distilled liquor until it reaching the 8 times weight of the herb and preserving the mother liquid separately; Cooling the distilled liquor at 0-4° C. for 12 hours and filtrating to collect the paeonol; dehydrating the wet paeonol by the cold wind and preserving the dry paeonol in airtight condition; The solid residual is mixed with the prepared rehmannia root, Chinese yam and Indian bread; the mixture is decocted in the water 8 times weight of the herbs for 3 times; each time continues 1.5 hours; the 3 piece of decocted liquor being mixed and added into the paeonol mother liquor; Concentrating the mixture to an extracturm with the relative density 1.10 at 80° C., centrifugating and concentrating the extracturm to extracturm I with the relative density 1;13 at 80° C.; the dogwood fruit and the alisma rhizome being crushed to coarse powder and added 8 times weight of 60% alcohol to extract refluxingly for 3 times; each time for 3 hours; Collecting the filtrated liquors and retrieving the alcohol, and then concentrating the filtrated liquor to the extracturm II with the relative density of 1.14 at 80° C.; mixing the extracturm 1 and II, then concentrating the mixture to a thick paste with the relative density of 1.30-1.35; dehydrating the thick paste in the vacuum, crushing the dry medicine to powder and then sifting the powder through the screen; Crushing the paeonol and mixing it with the sifted extracturm powder, adding right amount of supplementary materials and milling the mixture with the colloid mill; sifting the milled powder and putting it into 1000 soft capsules; each soft capsule is 1.0 g weight and equals to 3.0 g crude drug.

6. The soft capsule of medicine according to claim 4, wherein the method comprising:

| | |
|---|---|
| Prepared Rehmannia Root | 480 part-by-weight |
| Prepared Asiatic Cornelian Cherry Fruit | 240 part-by-weight |
| Tree peony Bark | 180 part-by-weight |
| Common Yan Rhizome | 240 part-by-weight |
| Indian Bread | 180 part-by-weight |
| Oriental Waterplantain Rhizome | 180 part-by-weight |
| Refined edible vegetable oil | sufficient quantum, |

soaking the root-bark of peony in the water 10 times of the herb for 2 hours, and then distilling the root-bark of peony by the water vapor for 10 hours; Collecting the distilled liquor until it reaching the 8 times weight of the herb and preserving the mother liquor separately; Cooling the distilled liquor at 0-4° C. for 12 hours and filtrating to collect the paeonol; dehydrating the wet paeonol by the cold wind and preserving the dry paeonol in airtight condition; mixing the solid residual with the prepared rehmannia root, Chinese yam and Indian bread; decocting the mixture is in the water 6 times weight of the herbs for 2 times; each time for 1 hours; mixing the 2 piece of decocted liquor and adding into the paeonol mother liquor; Concentrating the mixture to an extracturm with the relative density 1.10 at 80° C., centrifugating and concentrating the extracturm to extracturm I with the relative density 1.13 at 80° C.; crushing the dogwood fruit and the alisma rhizome to coarse powder and adding 6 times weight of 70% alcohol to extract refluxingly for 2 times; each time being 4 hours; Collecting the filtrated liquors and retrieving the alcohol, and then concentrating the filtrated liquor to the extracturm II with the relative density of 1.14 at 80° C.; mixing the extracturm 1 and II, then concentrating the mixture to a thick paste with the relative density of 1.30-1.35; dehydrating the thick paste in the vacuum, crushing the dry medicine to powder and then sifting the powder through the screen; crushing the paeonol and mixing it with the sifted extracturm powder, adding right amount of supplementary materials and milling the mixture with the colloid mill; sifting the milled powder and putting it into 1000 soft capsules; each soft capsule being 0.5 g weight and equals to 1.5 g crude drug.

7. A method for treating diabetes comprising administering the composition of medicine according to claim 1-3 to a patient in need thereof.

8. A method for treating hyperlipemia comprising administering the composition of medicine according to claim 1-3 to a patient in need thereof.

9. A method for preventing fatigue and enhancing the enduring of oxygen deficiency comprising administering the composition of medicine according to claim 1-3 to a patient in need thereof.
